(19) United States
(12) Patent Application Publication
(10) Pub. No.: US 2007/0179491 A1
(43) Pub. Date: Aug. 2, 2007

(54) Sensing needle for ablation therapy

(75) Inventors: Paul S. Kratoska, Brooklyn Park, MN (US); Ahmed Elmouelhi, Minneapolis, MN (US)

Correspondence Address: SHUMAKER & SIEFFERT, P. A., 1625 RADIO DRIVE, SUITE 300, WOODBURY, MN, 55125, US

(73) Assignee: Medtronic, Inc., Minneapolis, MN (US)

(21) Appl. No.: 11/343,921

(22) Filed: Jan. 31, 2006

(51) Int. Cl. A61B 18/18

(52) U.S. Cl. 606/32; 606/41

(57) ABSTRACT

The disclosure describes a method and a system that may be used to provide feedback on the progress of ablation therapy. The system includes a first needle that penetrates a target tissue to deliver radio frequency energy, with or without a conductive fluid, that heats and ablates the target tissue and a second needle that penetrates the target tissue and detects a tissue property indicative of the ablation progress. Temperature, impedance, or another parameter may be the tissue property detected and measured by the system. In addition, more than one sensor may be positioned on the first or second needle. The system may provide real-time monitoring of the tissue property or use the tissue property measurement to automatically terminate the ablation therapy. In particular, the system may be used to treat benign prostatic hypertrophy.

### TECHNICAL FIELD

The invention relates to medical devices and, more particularly, to devices for controlling therapy delivery.

### BACKGROUND

Tissue ablation is a commonly used surgical technique to treat a variety of medical conditions. Medical conditions may include excess tissue growth (such as benign prostatic hypertrophy), benign tumors, malignant tumors, destructive cardiac conductive pathways (such as ventricular tachycardia), and even sealing blood vessels during surgical procedures. Treatment for these medical conditions may include removing or destroying the target tissue, of which ablation is an appropriate solution.

Typically, ablation therapy involves heating the target tissue with a surgical instrument such as a needle or probe. The needle is coupled to an energy source which heats the needle, the target tissue, or both. Energy sources may cause ablation through radio frequency (RF) energy, heated fluids, impedance heating, or any combination of these sources. The needle may be presented to the target tissue during an open surgical procedure or through a minimally invasive surgical procedure.

As an example, benign prostatic hypertrophy (BPH) is a condition caused by the second period of continued prostate gland growth. This growth begins after a man is approximately 25 years old and may begin to cause health problems after 40 years of age. The prostate growth eventually begins to constrict the urethra and may cause problems with urination and bladder functionality. Minimally invasive ablation therapy may be used to treat this condition. A catheter is inserted into the urethra of a patient and directed to the area of the urethra adjacent to the prostate. An ablation needle is extended from the catheter and into the prostate. The clinician performing the procedure selects the desired ablation parameters and the needle heats the prostatic tissue. Ablation therapy shrinks the prostate to a smaller size that no longer interferes with normal urination and bladder functionality, and the patient may be relived of most problems related to BPH.

### SUMMARY

The disclosure is directed to a system that may be used to provide feedback on the progress of ablation therapy. Monitoring tissue surrounding tissue to be ablated or the ablated tissue may be useful feedback in accurately producing lesions of a certain size. As many ablation therapies are performed as minimally invasive procedures, a clinician cannot directly observe the ablation progress. Measuring one or more tissue property during the ablation procedure may allow the clinician to directly control the size of a lesion or automatically terminate ablative energy once a predetermined threshold has been reached. This feedback may also be included as a safety feature for ablation systems.

The system includes a first needle that penetrates a target tissue to deliver radio frequency energy that heats and ablates the target tissue and a second needle that penetrates the target tissue and detects a tissue property indicative of the ablation progress. The energy may be coupled with a conductive fluid to ablate the tissue. Temperature, impedance, or another property may be the tissue property detected and measured by the system. In addition, more than one sensor may be positioned on the first or second needle. The system may provide real-time monitoring of the tissue property or use the tissue property measurement to automatically terminate the ablation therapy. In particular, the system may be used to treat benign prostatic hypertrophy.

In one embodiment, this disclosure is directed to a method for providing feedback during tissue ablation that includes deploying a first needle and a second needle from a common catheter into a target tissue, wherein the first and second needles exit from one or more sides of the common catheter, delivering energy via the first needle to ablate at least a portion of the target tissue, and measuring a tissue property via the second needle.

In another embodiment, this disclosure is directed to a system that provides feedback during tissue ablation which includes a generator that generates energy to ablate at least a portion of a target tissue, a first needle that delivers the energy to the target tissue, a second needle that detects a tissue property, and a common catheter that houses at least a portion of each of the first needle and the second needle, wherein the first and second needles exit from one or more sides of the common catheter.

In an additional embodiment, this disclosure is directed to a device that provides feedback during tissue ablation that includes a first needle that delivers the energy to the target tissue, a second needle that detects a tissue property, and a common catheter that houses at least a portion of each of the first needle and the second needle, wherein the first needle exits a first opening in a side of the common catheter and the second needle exits a second opening of the side of the common catheter when the first and second needles are deployed.

In various embodiments, the device described in this disclosure may provide one or more advantages. The direct measurement of tissue properties during therapy may allow a clinician to produce accurate lesion sizes. These accurate sizes may reduce the number of ablation procedures needed to treat a patient and reduce the risk to inadvertently destroying non-target tissue. The measurements may also provide a closed feedback system in which the ablation treatment is completely automated based upon predetermined parameters. In this manner, the ablation treatment may be more consistent and independent of individual tissue consistencies or abnormalities. The system may also be able to treat much smaller target tissue due to direct measurements capable of quickly monitoring changes to tissue properties.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 is a conceptual diagram illustrating an example generator system in conjunction with a patient.

FIG. 2 is a side view of an example hand piece and connected catheter that delivers therapy to target tissue.

FIGS. 3A and 3B are cross-sectional side views of an example catheter tip in which a therapy needle exits to reach the target tissue.

FIGS. 4A and 4B are cross-sectional front views of an example catheter tip and exiting ablation and sensing needles.

FIGS. 5A, 5B, 5C and 5D are cross-sectional front views of exemplary ablation and sensing needles with varying sensing element configurations.

FIG. 6 is a conceptual diagram of ablation progress and detection with a sensing needle.

FIG. 7 is functional block diagram illustrating components of an exemplary generator system.

FIG. 8 is a flow diagram illustrating an example technique for automatically controlling tissue ablation with a sensing needle.

FIG. 9 is a flow diagram illustrating an example technique for a clinician to monitor tissue ablation with the aid of a sensing needle.

### DETAILED DESCRIPTION

This disclosure is directed to an ablation system that provides feedback regarding the progress of ablation therapy. Tissue ablation may be performed in an open surgical procedure or in a minimally invasive procedure. During a minimally invasive procedure, an ablation device is inserted into a patient until it reaches a target tissue. Since the target tissue cannot be visually inspected during treatment, the clinician usually selects a preferred lesion size or treatment time that estimates the end treatment point based upon the characteristics of the ablation device. The feedback system described herein may allow the clinician to be more precise in treating the tissue by monitoring the ablation progress. For example, measuring a temperature at a defined distance from the treatment site may enable a more accurate lesion size to be created in the target tissue.

The system includes a catheter that is introduced to a body cavity adjacent to a target tissue that is to be ablated. A first needle is extended, or deployed, from the catheter and penetrates into the target tissue. The first needle heats the target tissue to a temperature that causes tissue ablation. A second needle is also extended, or deployed, from the catheter and penetrates into the target tissue. The target tissue may be the same type of tissue or organ, or the target tissue may include multiple tissues or organs. The second needle detects a tissue property of the target tissue in real-time during the ablation therapy, where the tissue property may be tissue temperature or tissue impedance. In this manner, the second needle may provide feedback indicative of the ablation progress. This feedback may provide monitoring for the clinician to determine when to stop therapy or an automatic modification or termination control which modifies therapy once a threshold is reached.

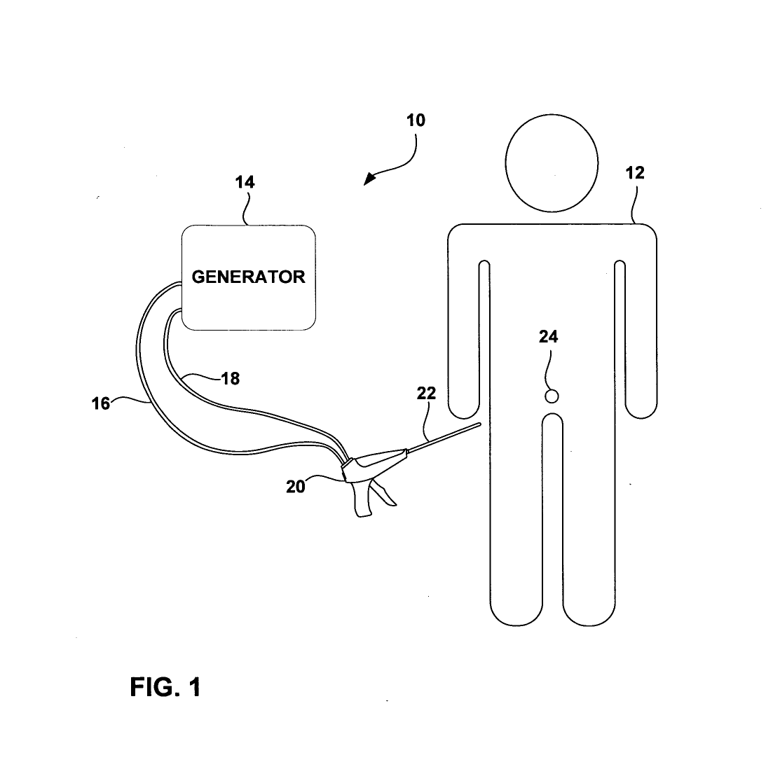

FIG. 1 is a conceptual diagram illustrating an example generator system in conjunction with a patient. As shown in the example of FIG. 1, system 10 may include a generator 14 that delivers therapy to treat a condition of patient 12. In this exemplary embodiment, generator 14 is a radio frequency (RF) generator that provides RF energy to heat tissue of the prostate gland 24. This ablation of prostate tissue destroys a portion of the enlarged prostate caused by, for example, benign prostatic hyperplasia (BPH). The RF energy is transmitted through electrical cable 16 to therapy device 20. The energy is then transmitted through a catheter 22 and is delivered to prostate 24 by a needle electrode (not shown in FIG. 1). In addition to the needle, a conductive fluid may be pumped out of delivery device 14, through tubing 18, into therapy device 20, and through catheter 22 to interact with the RF energy being delivered by the needle. This “wet electrode” may increase the effective heating area of the needle and increase therapy efficacy. A ground pad (not shown) is placed at the lower back of patient 12 to return the energy emitted by the needle electrode.

In the illustrated example, generator 14 is an RF generator that includes circuitry for developing RF energy from an included rechargeable battery or a common electrical outlet. The RF energy is produced within parameters that are adjusted to provide appropriate prostate tissue heating. The RF current is conveyed from generator 14 via electrical cable 16 which is connected to the generator. The conductive fluid is provided to the needle by a pump (not shown) also located within generator 14. In some embodiments, a conductive fluid may not be used in conjunction with the RF energy. This embodiment may be referred to as a “dry electrode” ablation system. Alternatively, other energy sources may be used in place of RF energy.

Therapy energy and other associated functions such as fluid flow are controlled via a graphic user interface located on a color liquid crystal display (LCD), or equivalent screen of generator 14. The screen may provide images created by the therapy software, and the user may interact with the software by touching the screen at certain locations indicated by the user interface. In this embodiment, no additional devices, such as a keyboard or pointer device, are needed to interact with the device. The touch screen may also enable device operation. In some embodiments, the device may require an access code or biometric authorization to use the device. Requiring the clinician to provide a fingerprint, for example, may limit unauthorized use of the system. Other embodiments of generator 14 may require input devices for control, or the generator may require manual operation an minimal computer control of the ablation therapy.

Connected to generator 14 are a cable 16 and a tube 18. Cable 16 conveys RF energy and tube 18 conducts fluid from generator 14 to therapy device 20. Cable 16 may also include wiring coupled to a second needle (not shown) that detects the tissue property. In other embodiments, a separate cable may include this sensing wiring. Tube 18 may carry conductive fluid and cooling fluid to the target tissue, or an additional tube (not shown) may carry the cooling fluid used to irrigate the urethra of patient 12.

Therapy device 20 may be embodied as a hand-held device as shown in FIG. 1. Therapy device 20 may include a trigger to control the start and stop of therapy. The trigger may be pressure sensitive, where increased pressure of the trigger provides an increased amount of RF energy or increase the fluid flow to the tissue of prostate 24. The trigger may also deploy the first and second needles into the target tissue. Attached to the distal end of therapy device 20 is a catheter 22. Catheter 22 may provide a conduit for both the RF energy and the fluid. Since the catheter 22 would be entering patient 12 through the urethra, the catheter may be very thin in diameter and long enough to reach the prostate in most any anatomical dimensions.

The end of catheter 22 may contain one or more electrodes for delivering RF current to the tissue of enlarged prostate 24. Catheter 22 may contain a first needle that is an electrode for penetrating into an area of prostate 24 from the urethra and a second needle for detecting tissue properties. More than one needle electrode or more than one detecting needle may be used in system 10. When RF energy is being delivered, the target tissue increases in temperature, which destroys a certain volume of tissue. This heating may last a few seconds or a few minutes, depending on the condition of patient 12. In some embodiments, the conductive fluid may exit small holes in the first needle and flow around the electrode. This conducting fluid, e.g., saline, may increase the effective heating area and decrease the heating time for effective treatment. Additionally, ablating tissue in this manner may enable the clinician to complete therapy by repositioning the needles a reduced number of times. The detecting needle may also increase ablation efficacy by accurately controlling the size of the lesion created by the ablation therapy. In this manner, patient 12 may require fewer treatment sessions to effectively treat BPH.

In some cases, therapy device 20 may only be used for one patient. Reuse may cause infection and contamination, so it may be desirable for the therapy device to only be used once. A feature on therapy device 20 may be a smart chip in communication with generator 14. For example, when the therapy device is connected to generator 14, the generator may request use information from the therapy device. If the device has been used before, generator 14 may disable all functions of the therapy device to prevent reuse of the device. Once therapy device 20 has been used, the smart chip may create a use a log to identify the therapy delivered and record that the device has been used. The log may include graphs of RF energy delivered to the patient, total RF energy delivered in terms of joules or time duration, error messages created, measure tissue properties, end lesion volume, or any other pertinent information to the therapy.

In other embodiments, catheter 22 may independently include the first and second needles such that different catheters may be attached to therapy device 20. Different catheters 20 may include different configurations of the first and second needles, such as lengths, diameters, number of needles, or sensors in the second needle. In this manner, a clinician may select the desired catheter 22 that provides the most efficacious therapy to patient 12.

While the example of system 10 described herein is directed toward treating BPH in prostate 24, system 10 may utilize a second needle for ablation feedback at any other target tissue of patient 12. For example, the target tissue may be polyps in a colon, a kidney tumor, esophageal cancer, uterine cancer tissue, or liver tumors. In any case, a second needle is included to provide feedback through the detection of a tissue property, such as tissue impedance or tissue temperature.

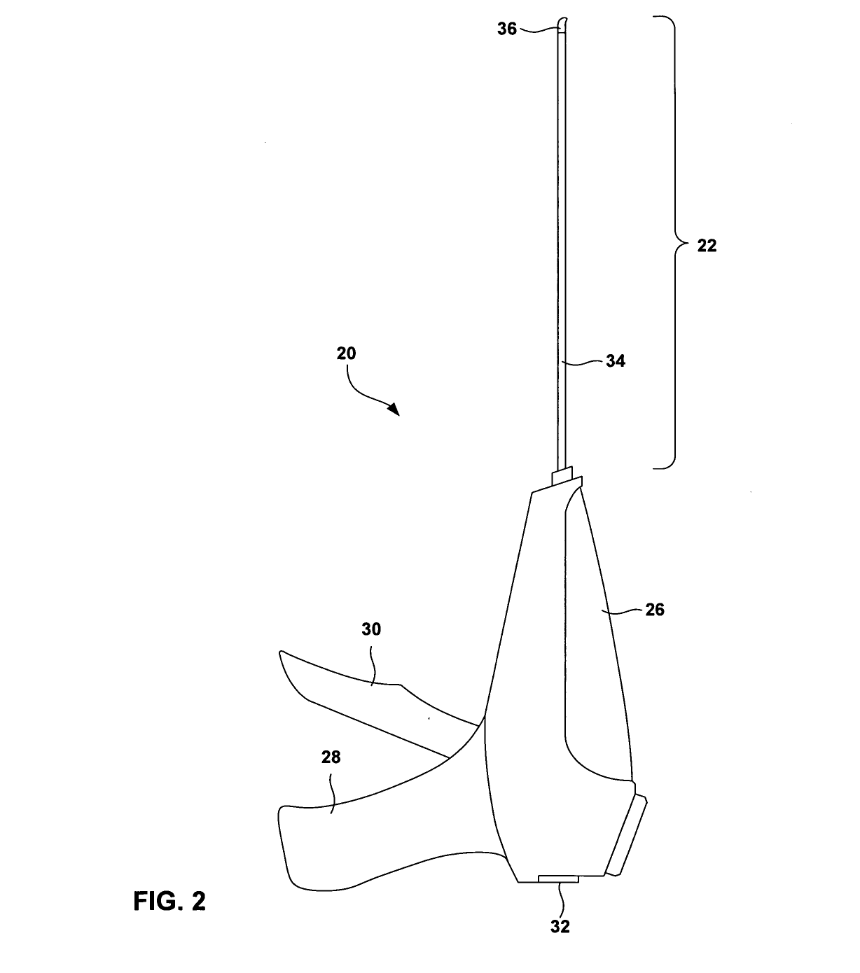

FIG. 2 is a side view of an example hand piece and connected catheter that delivers therapy to target tissue. As shown in FIG. 2, therapy device 20 includes housing 26 which is attached to handle 28 and trigger 30. A cystoscope (not shown), may be inserted though axial channel 32 and fitted within catheter 22. Catheter 22 includes shaft 34 and tip 36. A clinician holds handle 28 and trigger 30 to guide catheter 22 through a urethra. The clinician uses the cystoscope to view the urethra through tip 36 and locate a prostate for positioning the first and second needles (not shown) into prostate 24 from the tip. Once the clinician identifies correct placement for the needles, trigger 30 is squeezed toward handle 28 to extend the needles into prostate 24.

Housing 26, handle 28 and trigger 30 are constructed of a lightweight molded plastic such as polystyrene. In other embodiments, other injection molded plastics may be used such as polyurethane, polypropylene, high molecular weight polyurethane, polycarbonate or nylon. Alternatively, construction materials may be aluminum, stainless steel, a metal alloy or a composite material. In addition, housing 26, handle 28 and trigger 30 may be constructed of different materials instead of being constructed out of the same material. In some embodiments, housing 26, handle 28 and trigger 30 may be assembled through snap fit connections, adhesives or mechanical fixation devices such as pins or screws.

Shaft 34 of catheter 22 may be fixed into a channel of housing 26 or locked in place for a treatment session. Catheter 22 may be produced in different lengths or diameters with different configurations of needles or tip 36. A clinician may be able interchange catheter 22 with housing 26. In other embodiments, catheter 22 may be manufactured within housing 26 such that the clinician may have to use therapy device 20 as once medical device.

Shaft 34 is a rigid structure that is manufactured of stainless steel or another metal alloy and insulated with a polymer such as nylon or polyurethane. Alternatively, shaft 34 may be constructed of a rigid polymer or composite material. Shaft 34 includes one or more channels that house the first needle, the second needle, and a cystoscope. Tip 36 is constructed of an optically clear polymer such that the clinician may view the urethra during catheter 22 insertion. Shaft 34 and tip 36 may be attached with a screw mechanism, snap fit, or adhesives. Tip 36 also includes openings that allow the first and second needles to exit catheter 22 and extend into prostate 24.

In some embodiments, housing 26, handle 28, or trigger 30 may include dials or switches to control the deployment of the first and second needles in unison or independently. These controls may finely tune the ability of the clinician to tailor the therapy for patient 12. Housing 26 may also include a display that shows the clinician the tissue property used for feedback. For example, the temperature detected by the second needle may be displayed directly on therapy device 20 for easy viewing.

In other embodiments, shaft 34 and tip 36 may be configured to house more than one first needle or more than one second needle. For example, multiple first needles may be employed to treat a larger volume of tissue at one time. Alternatively, multiple second needles may be used to provide more accurate feedback relating to the ablation progress.

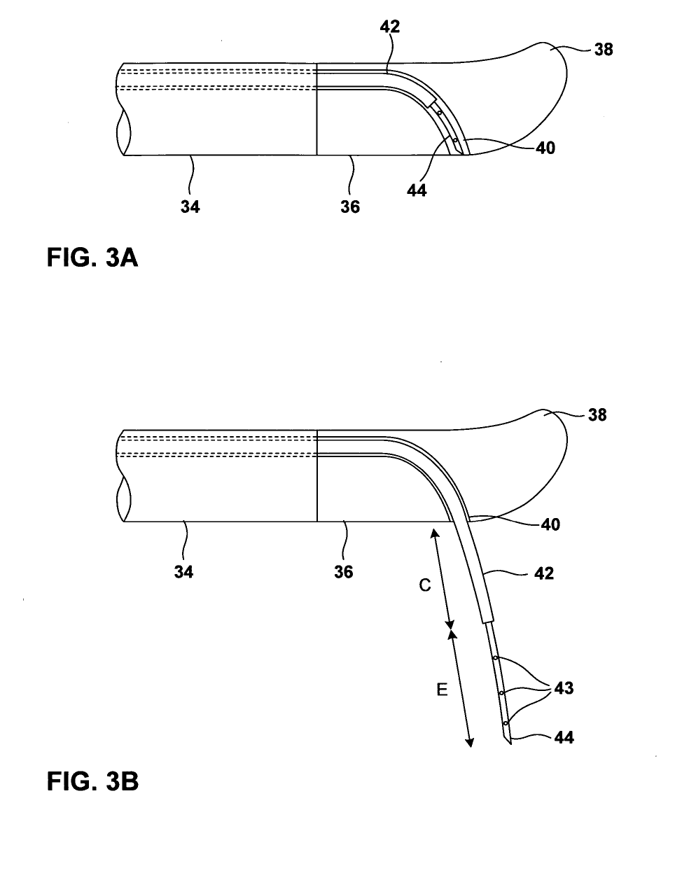

FIGS. 3A and 3B are cross-sectional side views of an exemplary catheter tip in which a therapy needle exits to reach the target tissue. As shown in FIG. 3A, shaft 34 is coupled to tip 36 at the distal end of catheter 22. Tip 36 includes protrusion 38 that aids in catheter insertion through the urethra. Tip 36 also includes channel 40 which allows first needle 44 to exit tip 36. First needle 44 is insulated with sheath 42, such that the exposed portion of first needle 44 may act as an electrode. The second needle (not shown) resides behind first needle 44 and cannot be seen in FIG. 3A.

Channel 40 continues from tip 36 through shaft 34. The curved portion of channel 40 in tip 36 deflects first needle 44 such that the first needle penetrates the target tissue from the side of catheter 22. The curvature of channel 40 may be altered to produce different entry angles of first needle 44 and the second needle. However, the needles should not extend beyond the distal end of tip 36. In other words, the needles may exit at or near the side of catheter 22, wherein the side is a lengthwise edge substantially facing the wall of the urethra. The wall of the urethra is a tissue barrier as it surrounds catheter 22. In some embodiments, the distal ends of first needle 44 or the second needle may stop at a point further from housing 26 than the distal end of tip 36.

As shown in FIG. 3B, first needle 44 has been deployed from tip 36 of catheter 22. The exposed length E of first needle 44 is variable by controlling the position of sheath 42. The covered length C of first needle 44 is that length of the first needle outside of tip 36 that is not delivering energy to the surrounding tissue. Exposed length E may be controlled by the clinician to be generally between 1 mm and 50 mm. More specifically, exposed length E may be between 12 mm and 22 mm. Covered length C may be generally between 1 mm and 50 mm. Specifically, covered length C may also be between 12 mm and 22 mm. Once first needle 44 and the second needle are deployed, the needles may be locked into place until the ablation therapy is completed.

First needle 44 is a hollow needle which allows conductive fluid, i.e., saline, to flow from generator 14 to the target tissue. First needle 44 includes multiple holes 43 which allow the conductive fluid to flow into the target tissue and increase the size of the needle electrode. The conductive fluid may also more evenly distribute the RF energy to the tissue to create more uniform lesions. In some embodiments, first needle 44 may also include a hole at the distal tip of the first needle. In other embodiments, first needle 44 may only include a hole at the distal tip of the first needle. Generator 14 may include a pump that delivers the conductive fluid at a predetermined flow rate, a flow rate adjusted by the clinician, or a flow rate determined automatically by sensors (such as the second needle).

Alternatively, first needle 44 may not deliver a conductive fluid to the target tissue. In this case, the first needle may be solid or hollow and act as a dry electrode. Delivering energy through first needle 44 without a conductive fluid may simplify the ablation procedure and reduce the cost of ablation therapy.

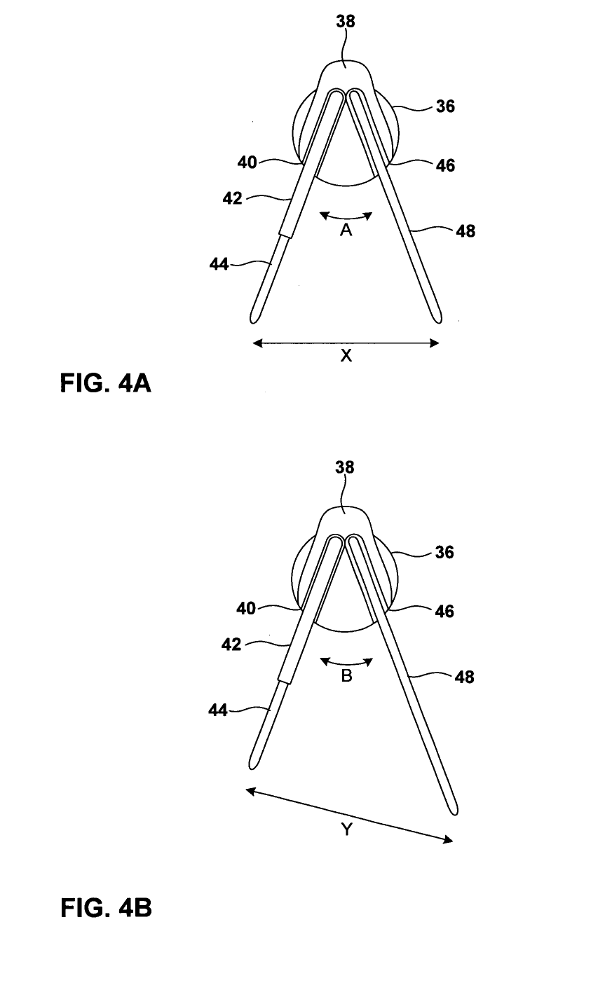

FIGS. 4A and 4B are cross-sectional front views of an example catheter tip and exiting ablation and sensing needles. As shown in FIG. 4A, first needle 44 and second needle 48 are deployed from tip 36 of catheter 22. First needle 44 is partially covered by sheath 42 and housed within channel 40. Second needle 48 is housed within channel 46 which mirrors the path of channel 40 shown in FIGS. 3A and 3B. Channels 40 and 46 may or may not be identical in diameter. First needle 44 and second needle 48 are deployed simultaneously and to the same extended length.

First needle 44 and second needle 48 may be constructed of similar materials or different materials. Exemplary materials may include stainless steel, nitinol, copper, silver, or an alloy including multiple metals. In any case, each needle may be flexible and conduct electricity to promote ablation and detection mechanisms. Second needle 48 may be hollow to include sensors or be formed around such sensors.

Second needle 48 is a detecting or sensing needle that is used for providing feedback regarding the ablation process. The tissue property detected by second needle 48 may be impedance, temperature, or another parameter indicative of a lesion produced by tissue ablation. Temperature may be detected by a sensor, such as a thermocouple or thermistor, housed within second needle 48. Additional temperature measurements may be provided by multiple sensors in second needle 48 or even one or more sensors within first needle 44. Generator 14 may measure the signal produced by the sensor and output a measure temperature of the tissue at that point. Impedance may be detected by a measurement between first needle 44 and second needle 48, or the second needle and the ground pad located on the back of patient 12. Increasing impedance is indicative of a great percentage of ablated tissue. The measurements provided by second needle 48 may be used in terminating the ablation therapy once a desired lesion is formed.

First needle 44 and second needle 48 exit tip 36 at angle A with respect to each other. Angle A may be varied by selecting different catheters 22 before the procedure. Generally, angle A is between 0 degrees and 120 degrees. More specifically, angle A is between 35 degrees and 50 degrees. In the preferred embodiment, angle A is approximately 42.5 degrees. While angle A is bisected by the midline of catheter 22, angle A may be offset to either side so that the needles do not form a symmetrical angle to the catheter.

The length first needle 44 and second needle 48 is deployed and angle A determines the distance X between the distal ends of each needle. Distance X may be varied such that second needle 48 is positioned at a distance to effectively provide feedback about the ablation progress. Generally, distance X is between 1 mm and 50 mm. More specifically, distance X may be between 6 mm and 20 mm. Preferably, distance X is approximately 13 mm. Distance X may be entered into generator 14 to accurately measure the tissue property of interest. For example, distance X may be useful when measuring the tissue impedance between first needle 44 and second needle 48.

In some embodiments, a sheath similar to sheath 42 may be included around second needle 48. The sheath may expose the desired length of second needle 48 and prevent fluids from entering channel 46.

As shown in FIG. 4B, first needle 44 and second needle 48 are extended at angle B with respect to each other. However, first needle 44 and second needle 48 have differing extended lengths. Second needle 48 is deployed at a longer length than first needle 44 to create a distance Y between the distal ends of each needle. In other embodiments, first needle 44 may be extended to a distance greater than second needle 48.

First needle 44 and second needle 48 may be deployed simultaneously using trigger 30 of therapy device 20. An internal mechanism may extend second needle 48 at a faster rate or limit the length of first needle 44 before limiting the length of the second needle 48. In either case, the clinician may control the length of each needle. Generator 14 may determine distance Y based upon the angle B and lengths of each needle, or the clinician may input the needle lengths into the generator. In other embodiments, first needle 44 and second needle 48 may have independent triggers 30 or other deployment mechanisms that allows the clinician to utilize two different lengths for the first and second needle. Increasing or decreasing distance Y may allow the clinician to accurately determine the size of a produced lesion.

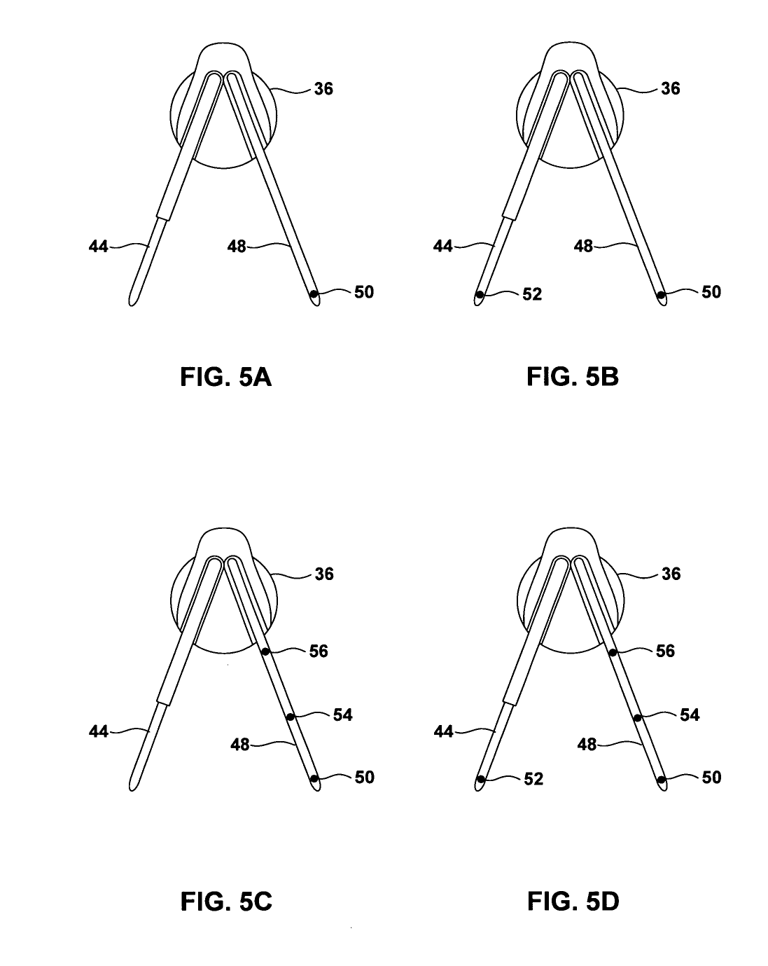

FIGS. 5A, 5B, 5C and 5D are cross-sectional front views of exemplary ablation and sensing needles with varying sensing element configurations. As shown in FIGS. 5A-5D, thermocouples are located at different positions of first needle 44 and second needle 48. These configurations may be available to the clinician by changing therapy device 20 or catheter 22. FIG. 5A shows thermocouple 50 located at the distal end of second needle 48. FIG. 5B shows thermocouple 50 at the distal end of second needle 48 and thermocouple 52 located at the distal end of first needle 44. Providing multiple thermocouples to obtain more than one temperature reading may allow a temperature gradient to be monitored between the sensors.

FIG. 5C is an example of three thermocouples 50, 54 and 56 located at various positions on second needle 48. Thermocouples 50, 54 and 56 may provide temperatures for multiple distances away from the source of ablation energy, first needle 44. FIG. 5D includes thermocouples 50, 54 and 56 on second needle 48 and thermocouple 52 on first needle 44. The configuration of FIG. 5D may allow a more accurate temperature profile of the lesion being produced by first needle 44. The clinician may desire more feedback to provide the more effective and precise treatment for patient 12.

In other embodiments, more or less thermocouples may be used to detect temperatures a various locations within prostate 24. In addition, sensors may include thermistors, a combination of thermistors and thermocouples, or any other temperature sensing elements. In some embodiments, infrared light or chemical sensors may be provided by second needle 48 to measure the temperature of the target tissue or lesion.

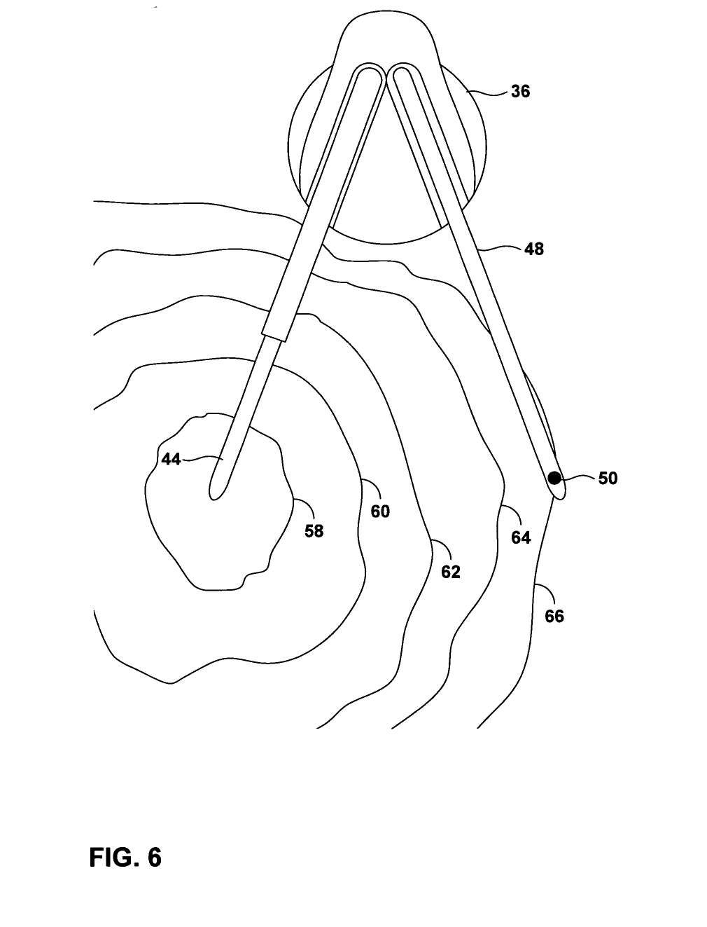

FIG. 6 is a conceptual diagram of ablation progress and detection with a sensing needle. As shown in FIG. 6, tip 36 of catheter 22 is inserted in the urethra. First needle 44 and second needle 48 are deployed into prostate 24, the target tissue. Second needle 48 includes thermocouple 50 for measuring the temperature of the tissue at that distance from first needle 44. Lesion perimeters 58, 60, 62, 64 and 66 are indicative of the lesion size after a certain duration of applying ablation energy to the tissue.

Once RF energy is delivered to first needle 44 by generator 14, the tissue immediately surrounding first needle 44 begins to increase in temperature. Once the tissue temperature reaches a threshold of approximately 80 degrees Celsius, the tissue is destroyed (ablated). The clinician desires to create a lesion radius equal to the distance between the distal end of first needle 44 and thermocouple 50 at the distal end of second needle 48. Therefore, once thermocouple 50 measures a temperature of 80 degrees Celsius, generator 14 will terminate ablation energy.

As an example, lesion perimeter 58 indicates a lesion size after 10 seconds and lesion perimeter 60 indicates the lesion size after 20 seconds of treatment. Lesion perimeter 62 is reached after 30 seconds, and lesion perimeter 64 is reached after 40 seconds. At the 40 second time point, thermocouple 50 may measure a temperature of approximately 76 degrees Celsius, so the RF energy is still delivered through first needle 44. After 50 seconds, lesion perimeter 66 is reached which triggers thermocouple 50 to measure a temperature of 80 degrees Celsius. Therefore, generator 14 terminates RF energy delivery to stop increasing the size of the lesion. Times provided in this example are only for illustrative purposes and may not be representative of actual ablation times. Lesions may increase in size at varying rates, dependent on RF energy, first needle 44 size or exposed length, and conductive fluid flow, if applicable.

In some embodiments, the temperature measured by thermocouple 50 may be used to approximate the size of the lesion. For example, a smaller lesion may be produced by terminating therapy once the temperature at thermocouple 50 is 70 degrees Celsius. Alternatively, a lesion of greater size may be produced by terminating RF energy once thermocouple 50 indicates a temperature of 86 degrees Celsius. In other embodiments, impedance measurements may similarly be used to approximate the size of a produced lesion. In any case, the feedback provided by thermocouple 50 may be useful to the clinician and patient 12.

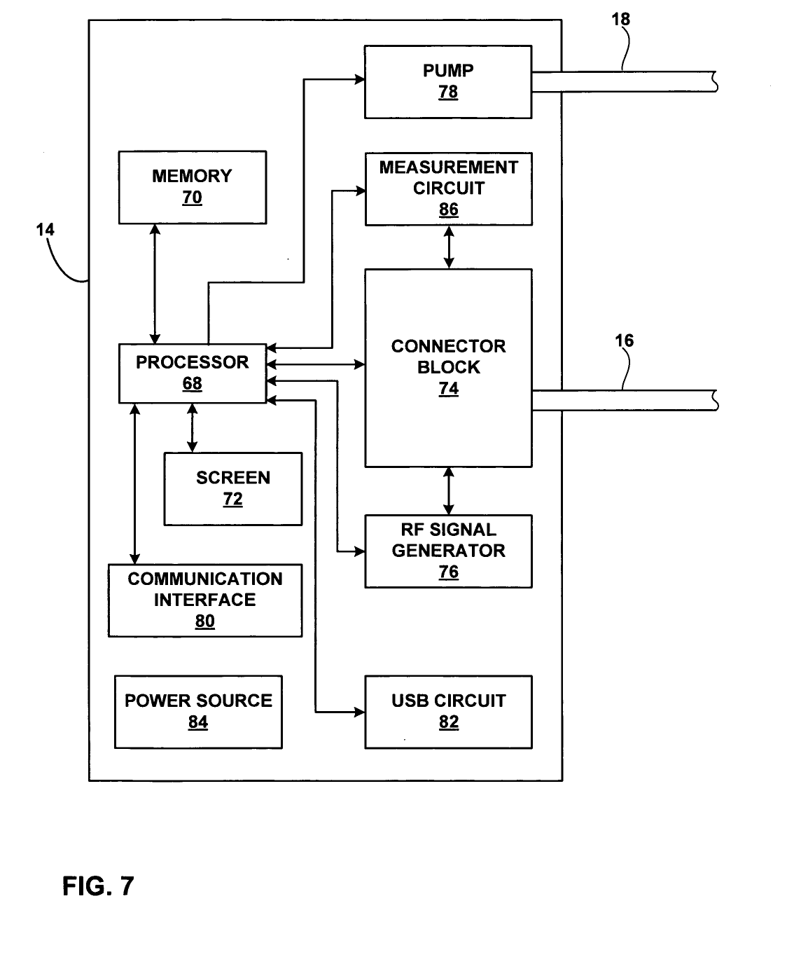

FIG. 7 is functional block diagram illustrating components of an exemplary generator system. In the example of FIG. 7, Generator 14 includes a processor 68, memory 70, screen 72, connector block 74, RF signal generator 76, measurement circuit 86, pump 78, telemetry interface 80, USB circuit 82, and power source 84. As shown in FIG. 7, connector block 74 is coupled to cable 16 for delivering RF energy produced by RF signal generator 76 and detecting tissue properties with measurement circuit 86. Pump 78 produces pressure to deliver fluid through tube 18.

Processor 68 controls RF signal generator 76 to deliver RF energy therapy through connector block 74 according to therapy parameter values stored in memory 70. Processor 68 may receive such parameter values from screen 72 or telemetry interface 80 or USB circuit 82. When signaled by the clinician, which may be a signal from therapy device 20 conveyed through connector block 74, processor 68 communicates with RF signal generator 76 to produce the appropriate RF energy. As needed, pump 78 provides fluid to irrigate the ablation site or provides fluid to the electrode during wet electrode ablation.

In a preferred embodiment, the RF signal generator may have certain performance parameters. In this exemplary case, the generator may provide RF energy into two channels with a maximum of 50 Watts per channel. The ramp time for a 50 Watt change in power may occur in less than 25 milliseconds. The output power may be selected in 1 Watt steps. The maximum current to be provided to the patient may be 1.5 Amps, and the maximum voltage may be 180 Volts.

Connector block 74 may contain an interface for a plurality of connections, not just the connection for cable 16. These other connections may include one for a return electrode, a second RF energy channel, or separate tissue property sensors. As mentioned previously, connector block 74 may be a variety of blocks used to diagnose or treat a variety of diseases. All connector blocks may be exchanged and connect to processor 68 for proper operation. Pump 78 may be replaceable by the clinician to replace a dysfunctional pump or use another pump capable of pumping fluid at a different flow rate.

Measurement circuit 86 may be configured to measure the impedance between first needle 44 and second needle 48, another impedance measurement, or temperature measurements from one or more sensors located in second needle 48 or first needle 44. In some embodiments, measurement circuit 86 may perform multiple sensing calculations to provide the clinician with impedance and temperature measurements of the target tissue.

Tissue properties, such as temperature measurements or impedance measurements, may also be monitored with measurement circuit 86 or processor 68 to track tissue property changes over time. Processor 68 may determine if the tissue property rate of change is faster or slower than predetermined rate of change thresholds. In addition, processor 68 may use current changes in the tissue property and ablation parameters to model or predict future tissue properties at certain time points. In this manner, the clinician may receive information such as the time remaining in a procedure, or processor 68 may change one or more ablation parameters automatically to effectively continue the ablation therapy.

During the use of a “wet electrode,” monitoring the tissue property change over time may be done during any combination of the delivery of RF energy and conductive fluid through needle 44. The tissue property change over time may be used to control fluid flow into prostate 24, RF power to needle 44, or time of ablation. In addition, the tissue property may be measured during initial fluid delivery to prostate 24 to monitor the fluid infusion before RF energy is delivered to ablate the prostate tissue. For example, the measured temperature may indicate successful fluid infusion by a decrease in temperature once room temperature conductive fluid is delivered to the tissue, and the temperature may increase with time as RF energy is delivered to ablate the tissue. In this manner, RF energy may be allowed to start as previously defined by the clinician or as the tissue property indicates that prostate 24 is ready for RF energy to be delivered. During the use of a “dry electrode,” monitoring the tissue property change over time may allow RF power or ablation time to be monitored.

Measurement circuit 86 may also perform calibration procedures to ensure accurate measurements of the tissue properties. The calibration of sensing elements may occur before every ablation treatment, during treatment, after every treatment, when generator 14 is turned on, or at any time the clinician desires to calibrate the sensors.

Processor 68 monitors the measured tissue property and may make modifications to the energy delivery or fluid delivery based upon the measured tissue property. The measured tissue property may be compared to a threshold set by the clinician or a predetermined program. In this manner, modifying energy delivery or fluid delivery may comprise any of starting, increasing, decreasing, or terminating either delivery. Modifying the energy or fluid delivery may be done individually or simultaneously.

Processor 68 may also control data flow from the therapy. Data such as RF energy produced, tissue properties measured from measurement circuit 86, and fluid flow may be channeled into memory 70 for analysis. Processor 68 may comprise any one or more of a microprocessor, digital signal processor (DSP), application specific integrated circuit (ASIC), field-programmable gate array (FPGA), or other digital logic circuitry. Memory 70 may include multiple memories for storing a variety of data. For example, one memory may contain therapy parameters, one may contain generator operational files, and one may contain measured therapy data. Memory 70 may include any one or more of a random access memory (RAM), read-only memory (ROM), electronically-erasable programmable ROM (EEPROM), flash memory, or the like.

Processor 68 may also send data to USB circuit 82 when a USB device is present to save data from therapy. USB circuit 82 may control both USB ports in the present embodiment; however, USB circuit 82 may control any number of USB ports included in generator 14. In some embodiments, USB circuit may be an IEEE circuit when IEEE ports are used as a means for transferring data.

The USB circuit may control a variety of external devices. In some embodiments, a keyboard or mouse may be connected via a USB port for system control. In other embodiments, a printer may be attached via a USB port to create hard copies of patient data or summarize the therapy. Other types of connectivity may be available through the USB circuit 82, such as internet access.

Communications with generator 14 may be accomplished by radio frequency (RF) communication or local area network (LAN) with another computing device or network access point. This communication is possible through the use of communication interface 80. Communication interface 80 may be configured to conduct wireless or wired data transactions simultaneously as needed by the clinician.

Generator 14 may communicate with a variety of devices to enable appropriate operation. For example, generator 14 may utilize communication interface 80 to monitor inventory, order disposable parts for therapy from a vendor, and download upgraded software for a therapy. For example, generator 14 may order a new catheter 22 is second needle 48 no longer measures tissue properties correctly. In some embodiments, the clinician may communicate with a help-desk, either computer directed or human staffed, in real-time to solve operational problems quickly. These problems with generator 14 or a connected therapy device may be diagnosed remotely and remedied via a software patch in some cases.

Screen 72 is the interface between generator 14 and the clinician. Processor 68 controls the graphics displayed on screen 72 and identifies when the clinician presses on certain portions of the screen 72, which is sensitive to touch control. In this manner, screen 72 operation may be central to the operation of generator 14 and appropriate therapy or diagnosis. Screen 72 may display the feedback measurements from second needle 48 during the ablation procedure. In a manual control mode, the clinician may monitor the measurements of one or more tissue properties to determine when to terminate the ablation treatment at that tissue location. In automatic mode, processor 68 may monitor the tissue property measurements and terminate the ablation treatment when a threshold is reached. A threshold may also be used in manual control mode as a safety mechanism.

Power source 84 delivers operating power to the components of generator 14. Power source 84 may utilize electricity from a standard 115 Volt electrical outlet or include a battery and a power generation circuit to produce the operating power. In some embodiments, the battery may be rechargeable to allow extended operation. Recharging may be accomplished through the 115 Volt electrical outlet. In other embodiments, traditional batteries may be used.

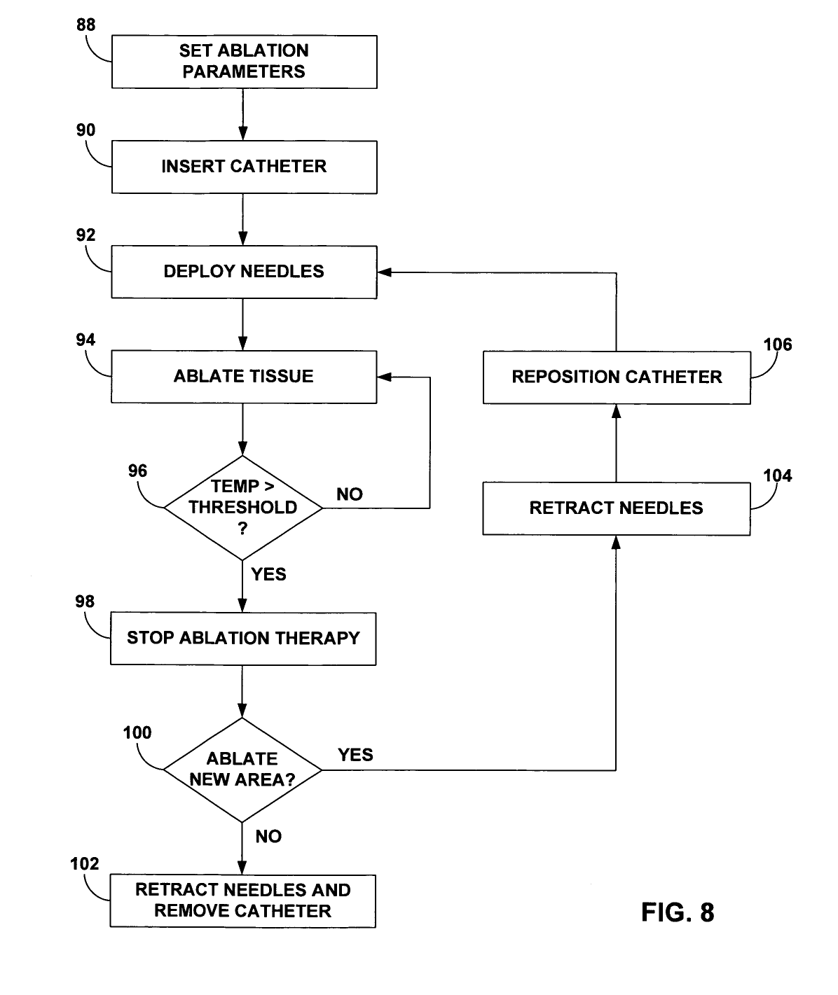

FIG. 8 is a flow diagram illustrating an example technique for automatically controlling tissue ablation with a sensing needle. As shown in FIG. 8, second needle 48 is used in a closed feedback system for controlling ablation therapy. The clinician sets ablation parameters in generator 14 (88). Ablation parameters may include RF power, needle lengths, or other parameters related to the therapy. Selecting a desired catheter 22 configuration may be an ablation parameter as well. The clinician next inserts catheter 22 into the urethra of patient 12 until tip 36 is correctly positioned adjacent to prostate 24 (90). The clinician may use a cystoscope within catheter 22 to guide the catheter. Once correctly positioned, the clinician deploys first needle 44 and second needle 48 into prostate 24 (92).

The clinician starts tissue ablation by pressing a button on generator 14 or therapy device 20 (94). Conductive fluid may or may not be delivered by first needle 44. Generator 14 monitors the temperature measured at second needle 48 and compares the temperature to a predetermined threshold temperature (96). For example, the threshold may be 80 degrees Celsius, which is indicative of ablated tissue at that location. If the temperature is not greater than the threshold, generator 14 continues ablating tissue (94). If the temperature is greater than the threshold, generator 14 automatically terminates the ablation therapy (98).

If the clinician does not want to ablate a new area of prostate 24 (100), the clinician retracts needles 44 and 48 and removes catheter 22 from patient 12 (102). If the clinician desires to ablate more tissue, the clinician retracts needles 44 and 48 (104), repositions catheter 22 adjacent to the new tissue area (106), and deploys the needles once more (92). Ablation may begin again to treat more tissue (94).

In some embodiments, generator 14 may monitor multiple temperatures and compare each temperature to a respective threshold. Generator 14 may terminate RF energy when all thresholds are reached, when a certain number of thresholds are reaches, or when only one threshold is reached. In other embodiments, impedance or another tissue property may be monitored to control the therapy.

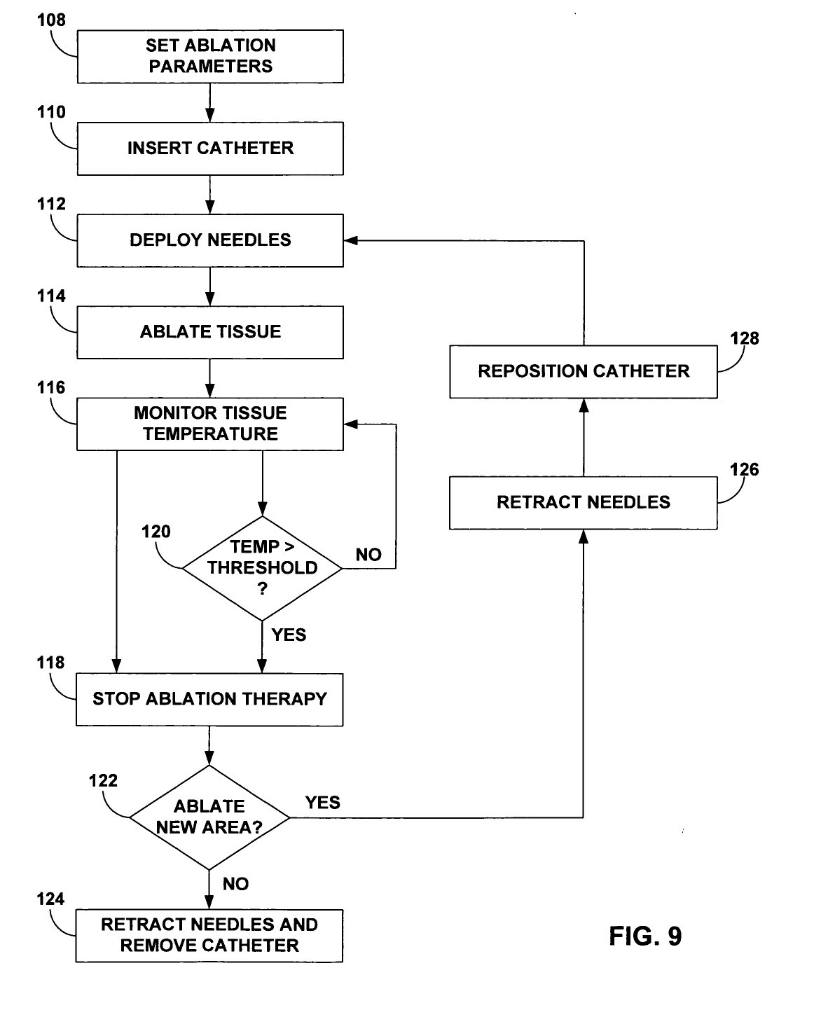

FIG. 9 is a flow diagram illustrating an example technique for a clinician to monitor tissue ablation with the aid of a sensing needle. FIG. 9 may be similar to the technique described in FIG. 8. As shown in FIG. 9, the clinician sets ablation parameters in generator 14 (108). Ablation parameters may include RF power, needle lengths, or other parameters related to the therapy. Selecting a desired catheter 22 configuration may be an ablation parameter as well. The clinician next inserts catheter 22 into the urethra of patient 12 until tip 36 is correctly positioned adjacent to prostate 24 (110). The clinician may use a cystoscope within catheter 22 to guide the catheter. Once correctly positioned, the clinician deploys first needle 44 and second needle 48 into prostate 24 (112).

The clinician starts tissue ablation by pressing a button on generator 14 or therapy device 20 (114). Conductive fluid may or may not be delivered by first needle 44. The clinician monitors the temperature measured at second needle 48 (116) and stops the ablation therapy once the clinician is satisfied with the temperature being measured (118). Concurrently, generator 14 monitors the temperature measured at second needle 48 and compares the temperature to a predetermined threshold temperature as a safety mechanism for the therapy (120). For example, the threshold may clinician set or permanently fixed to 85 degrees Celsius, which is indicative of well ablated tissue at that location. Generator 14 may include a fixed upper limit to the threshold temperature to avoid clinician mistakes. If the temperature is not greater than the threshold, generator 14 continues to allow the clinician to monitor the tissue temperature (116). If the temperature is greater than the threshold, generator 14 automatically terminates the ablation therapy (118). If the threshold has been reached, generator 14 may not allow the clinician to redeliver RF energy until needles 44 and 48 are retracted.

If the clinician does not want to ablate a new area of prostate 24 (122), the clinician retracts needles 44 and 48 and removes catheter 22 from patient 12 (124). If the clinician desires to ablate more tissue, the clinician retracts needles 44 and 48 (126), repositions catheter 22 adjacent to the new tissue area (128), and deploys the needles once more (112). Ablation may begin again to treat more tissue (114).

In some embodiments, the clinician and generator 14 may monitor multiple temperatures and compare each temperature to a respective threshold. Generator 14 may terminate RF energy for safety when all thresholds are reached, when a certain number of thresholds are reaches, or when only one threshold is reached. In other embodiments, impedance or another tissue property may be monitored by the clinician and generator 14 to control the therapy. Alternatively, the clinician may disable the safety threshold feature such that the ablation progress is completely manual and dependent upon clinician termination of RF energy.

Various embodiments of the described invention may include processors that are realized by microprocessors, Application-Specific Integrated Circuits (ASIC), Field-Programmable Gate Arrays (FPGA), or other equivalent integrated logic circuitry. The processor may also utilize several different types of storage methods to hold computer-readable instructions for the device operation and data storage. These memory and storage media types may include a type of hard disk, random access memory (RAM), or flash memory, e.g. CompactFlash or SmartMedia. Each storage option may be chosen depending on the embodiment of the invention. Generator 14 may contain permanent memory or a more portable removable memory type to enable easy data transfer for offline data analysis.

The preceding specific embodiments are illustrative of the practice of the invention. It is to be understood, therefore, that other expedients known to those skilled in the art or disclosed herein may be employed without departing from the invention or the scope of the claims.

Many embodiments of the invention have been described. Various modifications may be made without departing from the scope of the claims. These and other embodiments are within the scope of the following claims.

1. A method for providing feedback during tissue ablation, the method comprising:
deploying a first needle and a second needle from a common catheter into a target tissue, wherein the first and second needles exit from one or more sides of the common catheter;
delivering energy via the first needle to ablate at least a portion of the target tissue; and
measuring a tissue property via the second needle.

2. The method of claim 1, further comprising delivering a conductive fluid to the target tissue via the first needle.

3. The method of claim 2, further comprising moving the fluid through a plurality of holes in the first needle.

4. The method of claim 1, wherein the first and second needles do not extend beyond a distal tip of the common catheter.

5. The method of claim 1, wherein the first needle is deployed to a first length and the second needle is deployed to a second length.

6. The method of claim 5, wherein the first length is different from the second length.

7. The method of claim 1, wherein the tissue property is temperature.

8. The method of claim 7, further comprising measuring a temperature difference between the first needle and the second needle.

9. The method of claim 7, further comprising measuring a temperature change over time.

10. The method of claim 1, wherein the tissue property is impedance.

11. The method of claim 10, further comprising measuring an impedance change over time.

12. The method of claim 1, further comprising measuring a second tissue property at a second location of the second needle.

13. The method of claim 1, further comprising measuring a second tissue property via the first needle.

14. The method of claim 1, further comprising displaying the tissue property measurement to a user.

15. The method of claim 1, wherein delivering energy via the first needle is at least partially controlled by an insulated sleeve covering a portion of the first needle.

16. The method of claim 1, further comprising modifying at least one of fluid delivery and energy delivery when the tissue property reaches a threshold.

17. The method of claim 16, wherein modifying comprises terminating at least one of fluid delivery and energy delivery when the tissue property reaches the threshold.

18. The method of claim 1, further comprising retracting the first needle and the second needle after at least a portion of the target tissue is ablated.

19. The method of claim 1, wherein the target tissue is a prostate.

20. A system that provides feedback during tissue ablation, the system comprising:
a generator that generates energy to ablate at least a portion of a target tissue;
a first needle that delivers the energy to the target tissue;
a second needle that detects a tissue property; and
a common catheter that houses at least a portion of each of the first needle and the second needle, wherein the first and second needles exit from one or more sides of the common catheter.

21. The system of claim 20, further comprising a pump to deliver a conductive fluid to the target tissue via the first needle.

22. The system of claim 21, wherein the first needle comprises a plurality of holes for the fluid to pass through.

23. The system of claim 20, wherein the first and second needles do not extend beyond a distal tip of the common catheter.

24. The system of claim 20, wherein the first needle is deployed to a first length and the second needle is deployed to a second length.

25. The system of claim 24, wherein each of the first and second lengths are between 1 mm and 50 mm.

26. The system of claim 20, wherein a distance between a tip of the first needle and a tip of the second needle is between 1 mm and 50 mm.

27. The system of claim 20, wherein an angle formed between the first needle and the second needle is between 0 degrees and 120 degrees.

28. The system of claim 27, wherein the angle formed between the first needle and the second needle is between 35 degrees and 50 degrees.

29. The system of claim 20, wherein the second needle comprises a temperature sensor to detect the tissue property of temperature.

30. The system of claim 29, wherein multiple temperature sensors are located at different positions along the second needle.

31. The system of claim 29, wherein the first needle comprises a temperature sensor to detect the tissue property of temperature.

32. The system of claim 20, wherein the tissue property of impedance is detected between the first and second needles.

33. The system of claim 20, further comprising a user interface that displays a tissue property measurement to a user.

34. The system of claim 20, wherein the first needle is at least partially covered by an insulated sleeve.

35. The system of claim 20, wherein the generator modifies energy generation when the tissue property reaches a threshold.

36. The system of claim 21, wherein the pump modifies fluid delivery when the tissue property reaches a threshold.

37. The system of claim 20, wherein the target tissue is a prostate.

38. A device that provides feedback during tissue ablation, the device comprising:
a first needle that delivers the energy to the target tissue;
a second needle that detects a tissue property; and
a common catheter that houses at least a portion of each of the first needle and the second needle, wherein the first needle exits a first opening in a side of the common catheter and the second needle exits a second opening of the side of the common catheter when the first and second needles are deployed.

39. The device of claim 38, wherein the first needle comprises a plurality of holes that deliver a conductive fluid to the target tissue.

40. The device of claim 38, wherein the first and second needles do not extend beyond a distal tip of the common catheter.

41. The device of claim 38, wherein a distance between a tip of the first needle and a tip of the second needle is between 1 mm and 50 mm.

42. The device of claim 38, wherein an angle formed between the first needle and the second needle is between 0 degrees and 120 degrees.

43. The device of claim 42, wherein the angle formed between the first needle and the second needle is between 35 degrees and 50 degrees.

44. The device of claim 43, wherein the second needle comprises at least one temperature sensor to detect the tissue property of temperature.

45. The device of claim 44, wherein the first needle comprises a temperature sensor to detect the tissue property of temperature.

46. The device of claim 38, wherein the first and second needles detect the tissue property of impedance.
